(19) United States
(12) Patent Application Publication
(10) Pub. No.: US 2009/0317886 A1
(43) Pub. Date: Dec. 24, 2009

(54) Apparatus and Methods for Separating, Concentrating and Isolating Algae

(75) Inventor: W. Shannon Hamilton, Klamath Falls, OR (US)

Correspondence Address: HANCOCK HUGHEY LLP, P.O. BOX 1208, SISTERS, OR, 97759, US

(73) Assignee: KVB, LLC, Klamath Falls, OR (US)

(21) Appl. No.: 12/144,213

(22) Filed: Jun. 23, 2008

(51) Int. Cl. C12N 13/00; C12M 1/00

(52) U.S. Cl. 435/173.9; 435/283.1

(57) ABSTRACT

Apparatus and method to efficiently separate A. flos aquae from M. aeruginosa are described so that A. flos aquae may be processed for human consumption. A separation/concentration/isolation tank has a vertically oriented axis with an open top. The tank wall is at least partly transparent or translucent so that sunlight penetrates the tank wall and into the tank interior. The tank contains an aqueous solution containing both A. flos aquae and, typically, M. aeruginosa. The tank is exposed to the light source and as the organisms carry on metabolic processes, A. flos aquae and M. aeruginosa vertically stratify in the tank by virtue of the different buoyancy responses of the two organisms when exposed to the same environmental conditions. This results in the A. flos aquae being physically separated from M. aeruginosa and concentrated in the tank. The desired portion of the water-that is, the water containing concentrated A. flos aquae, may be removed from the tank for further concentration and purification.

### FIELD OF THE INVENTION

This invention relates to apparatus and methods for separating and isolating different species of algae in order to concentrate desired species of algae in an aliquot, and for removing undesired organisms from the aliquot, and more specifically, to apparatus and method for concentrating desired species of cyanobacteria so that the desired species may be harvested, and for removing undesired species from the same aliquot.

### BACKGROUND

Cyanobacteria are single-celled, primitive, prokaryotic organisms that resemble bacteria in their internal cell organization. Cyanobacteria sometimes joined together in colonies or filaments. Cyanobacteria are among the oldest known living organisms and, with bacteria, belong to the kingdom Monera. Fossil evidence found in rocks suggests that forms of cyanobacteria existed up to 3.5 billion years ago. They are widely distributed in aquatic habitats, on the damp surfaces of rocks and trees, and in the soil. Some can fix nitrogen and thus are necessary to the nitrogen cycle, while others follow a symbiotic existence—for example, living in association with fungi to form lichens.

Cyanobacteria may be found in a variety of different habitats, ranging from oceans and fresh water to soil. Most are found in fresh water, while others thrive in salt water habitats, in damp soil, or even temporarily moistened rocks in deserts. Cyanobacteria include unicellular and colonial species. Colonies occur in several forms, including filaments, sheets and hollow balls. Some filamentous colonies demonstrate the ability to differentiate into several different cell types: vegetative cells, which define the normal, photosynthetic cells that are formed under favorable growing conditions; akinetes, which define climate-resistant spores that may form when environmental conditions become harsh; and heterocyst, thick-walled cells that contain the enzyme nitrogenase, which is known to be vital for nitrogen fixation. Heterocysts may also form under the anoxic environmental conditions wherever nitrogen is necessary. Each individual cell of a cyanobacterium typically has a thick, gelatinous cell wall. Many cyanobacteria are capable of forming motile filaments, called hormogonia, that travel away from the main biomass to bud and form new colonies elsewhere. The cells in a hormogonium are often thinner than in the vegetative state, and the cells on either end of the motile chain may be tapered. Cyanobacterium lack flagella, but hormogonia may be motile by gliding along surfaces. In water columns some cyanobacteria float by forming gas vesicles.

Cyanobacteria have an elaborate and highly organized system of internal membranes which function in photosynthesis. Photosynthesis in cyanobacteria generally uses water as an electron donor and produces oxygen as a by-product, though some species may also use hydrogen sulfide as occurs among other photosynthetic bacteria. In most forms the photosynthetic machinery is embedded into folds of the cell membrane, called thylakoids. Due to their ability to fix nitrogen in aerobic conditions, cyanobacteria are often found in symbiotic relationships with a number of other groups of organisms such as fungi, corals, angiosperms, and others.

Some species of cyanobacteria are used as dietary supplements for human consumption. In particular, the blue green algae commonly referred to as “Klamath Blue Green Algae” (Aphanizomenon flos aquae, Family—Nostoceaceae; Species—Flos-Aquae; Phylum—Cyanophyta; Class—Myxophycae; Order—Nostocales; and Genus—Aphanizomenon). Although individual A. flos aquae cells are typically elliptical to rectangular in shape with the largest dimension being 20 microns, A. flos aquae usually exists in the form of needle-like cell clusters up to a millimeter or so in length. A. flos aquae flourishes naturally in Oregon's Upper Klamath Lake in late summer and early fall in quantities sufficient to allow commercial harvesting. This situation is believed to be unique and the only location where A. flos aquae is harvested for human consumption. A. flos aquae go dormant in the winter time when the lake freezes, but blooms in the summer and fall allowing commercial harvesting.

It is known that Native Americans gathered the blue green algae from Upper Klamath Lake in porous cloth bags. The filled bags containing water and blue green algae were allowed to drain, and the concentrated algal mass was then formed into large flat cakes on the sand and dried in the sun. As the blue-green algae gelled, it was smoothed by hand and marked off into squares. When most of the water evaporated or seeped into the sand, the squares were pulled up, dried further on mats and cut into brittle cakes for consumption.

A. flos aquae from Klamath lake green algae has been shown to be very nutritionally dense, offering an abundance and complexity of bio-nutrients. Specifically, A. flos aquae contain 20 antioxidants, 68 minerals and 70 trace elements, all amino acids (essential and non-essential) and important enzymes. In particular, it is its high nutrient density and the synergistic effect of the trace nutrients that it contains that makes the blue green A. flos aquae algae a legitimate superfood. It offers a maximum of important micronutrients and simultaneously a minimum of calories. As such it is one of the most concentrated natural foodstuffs known today. More information about Klamath lake A. flos aquae is available at www.klamathvalley.com, a web site operated by the assignee of the present patent application.

A. flos aquae is frequently used for human consumption either in the form of a dried powder material that is prepared directly from harvested liquid, which under conventional harvesting techniques contains from about 3 to 8% by weight of A. flos aquae, as a dried, water-soluble powder made from the harvested liquid, usually after the cell wall has been compromised in some way to allow the materials inside the cell to dissolve in the water. An important economic aspect in the cost of making either the powder is the machine time and energy required to obtain dry material from the wet starting material. Another cost factor that enters is that of freezing and storing the harvested liquid if it cannot be processed within 48 hours or so from harvesting. Consequently, there is great value in a low-cost method for increasing the A. flos aquae concentration prior to drying or freezing.

Moreover, under some lake and weather conditions, the A. flos aquae harvested from Upper Klamath Lake may be contaminated with Microcystis aeruginosa (referred to herein as “M. aeruginosa”), a cyanobacteria that synthesizes hepatotoxins known as microcystins, which can be responsible for acute poisonings and which has been implicated in the promotion of liver cancer at sublethal concentrations. Good Manufacturing Practice (GMP) as well as Oregon law prohibits the use of A. flos aquae for human consumption if the A. flos aquae contains in excess of one part per million (PPM) of microcystin in the final product. M. aeruginosa is a cyanobacterium that roughly spherical in shape and up to 100 microns in diameter. It often occurs in small, spherical clumps from 250 to 400 microns in diameter.

Under current commercial harvesting techniques, A. flos aquae are separated from M. aeruginosa in a cone filter separator. This method of separating the organisms is not highly efficient, and because there is a need for reducing the level of mycrocystin to less than 1 PPM, the losses in terms of A. flos aquae that is discarded are very high. There is a need, therefore, for a more effective and efficient apparatus and method for separating A. flos aquae from M. aeruginosa.

The present invention relates to a novel apparatus and method to efficiently separate A. flos aquae from M. aeruginosa so that the target species, A. flos aquae may be processed for human consumption. The apparatus is defined by a separation and concentration tank that has a wall that is transparent or translucent so that sunlight (or light from other sources such as artificial light having desired characteristics) penetrates the tank wall and into the tank interior. The tank (or tanks) has an open top and is filled with water that contains both A. flos aquae and, typically, M. aeruginosa. The tanks are exposed to the light source and as the organisms carry on metabolic processes, A. flos aquae and M. aeruginosa, which are exposed to identical environmental conditions in the tank, demonstrate different buoyancy responses. This results in the A. flos aquae being physically separated from M. aeruginosa in the tank as the two different organisms stratify in the tank as they rise in the vertical direction under buoyant forces, thereby concentrating the A. flos aquae. The desired portion of the water—that is, the water containing concentrated A. flos aquae, may be removed from the tank for further concentration and purification, ultimately being processed for human consumption. The water from the tank that contains M. aeruginosa may be discarded or otherwise processed.

### BRIEF DESCRIPTION OF THE DRAWINGS

The invention will be better understood and its numerous objects and advantages will be apparent by reference to the following detailed description of the invention when taken in conjunction with the following drawings.

FIG. 1 is a perspective view of an exemplary installation of equipment used according to the present invention.

FIG. 2 is a perspective view of an illustrated embodiment of a separation tank according to the presenting invention.

FIG. 3 is an elevation view of the separation tank shown in FIG. 2, illustrating selected components.

FIG. 4 is a cross sectional view taken along the line 4-4 of FIG. 3.

### DETAILED DESCRIPTION OF THE PREFERRED EMBODIMENTS

Both A. flos aquae and M. aeruginosa have subcellular structures—gas vesicles—that provide the cells with buoyancy. These gas vacuoles have been widely studied and among other properties, allow cells to float at the surface of water environments and can be used to otherwise position the cells under optimal light and oxygen conditions for growth. The structural shape, despite variations in width and length, is well conserved among different groups of organisms. Formed solely of proteins, the gas vacuoles are typically spindle-shaped cylinders with conical end caps. Individual vacuoles cluster into gas vacuoles that are visible under phase-contrast microscopy.

The buoyancy of both A. flos aquae and M. aeruginosa have been studied under a variety of conditions, and while both organisms demonstrate different responses to different environmental conditions—depending for example on factors such as light exposure, nutrient density and composition, and water temperature—the present invention relies on the fact that both A. flos aquae and M. aeruginosa exhibit different buoyancy responses in the same environmental conditions. Stated another way, when populations of both A. flos aquae and M. aeruginosa are exposed to identical environmental conditions, the two organisms exhibit different buoyancy characteristics and responses. Even more particularly, when an aliquot defined by a mixed aqueous solution containing both A. flos aquae and M. aeruginosa is exposed to light, the A. flos aquae and M. aeruginosa have different buoyancy responses.

This is due to many different factors, but one physical manifestation of the different buoyancy responses is that the two organisms tend to stratify in aqueous solution in the aliquot. This stratification is believed to be due at least in part to the fact that gas vacuoles in M. aeruginosa colonies are approximately spherical and much smaller than the thread-like colonies of A. flos aquae. The present invention relies upon the physical manifestations of this buoyancy characteristics, which in separation vessels fabricated according to the invention allows for separation of A. flos aquae from M. aeruginosa on a commercial basis.

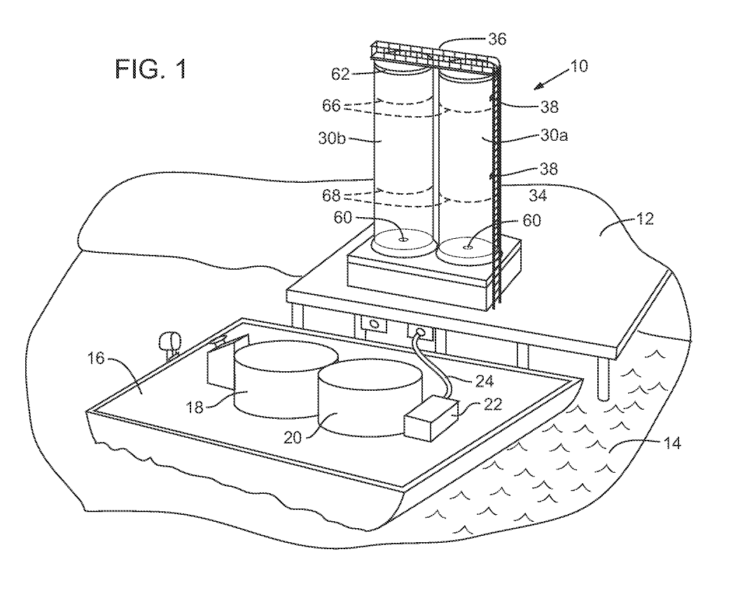

With reference now to FIG. 1, a separation apparatus 10 according to the present invention is shown as it might be used in an exemplary installation. Thus, separation apparatus 10 is depicted on a shoreside facility defined by dock 12, which is on the shore of a lake 14. A harvesting barge 16 is moored next to the dock in a position such that contents of the barge may be discharged from the barge into the separation apparatus. More specifically, as detailed below, harvesting barge 16 contains quantities of water in holds such as tanks 18 and 20. The water in tanks 18 and 20 contains desired organisms (A. flos aquae), and also undesired organisms (M. aeruginosa). The water from the tanks is pumped with pump 22 through transfer hose 24 into a distribution plumbing system that leads to the separation apparatus 10.

Separation apparatus 10 is defined by one or more cylindrical tanks, such as tanks 30a and 30b in FIG. 1, which as detailed below define separation, concentration and isolation vessels. Each tank is preferably relatively tall compared to its diameter—a preferred height to diameter ratio is greater than about 3 to 1, although this may be varied. Two tanks 30a and 30b are shown in FIG. 1 and the capacity of each tank is typically around 5,000 gallons, although the capacity of the tank may be varied widely. It will be readily understood that many tanks may be combined into a tank farm for purposes of the present invention. A ladder 34 provides access to a gangway 36 that may be used by workers during operation and for maintenance. The ladder 34 is attached to the tank 30 with brackets 38.

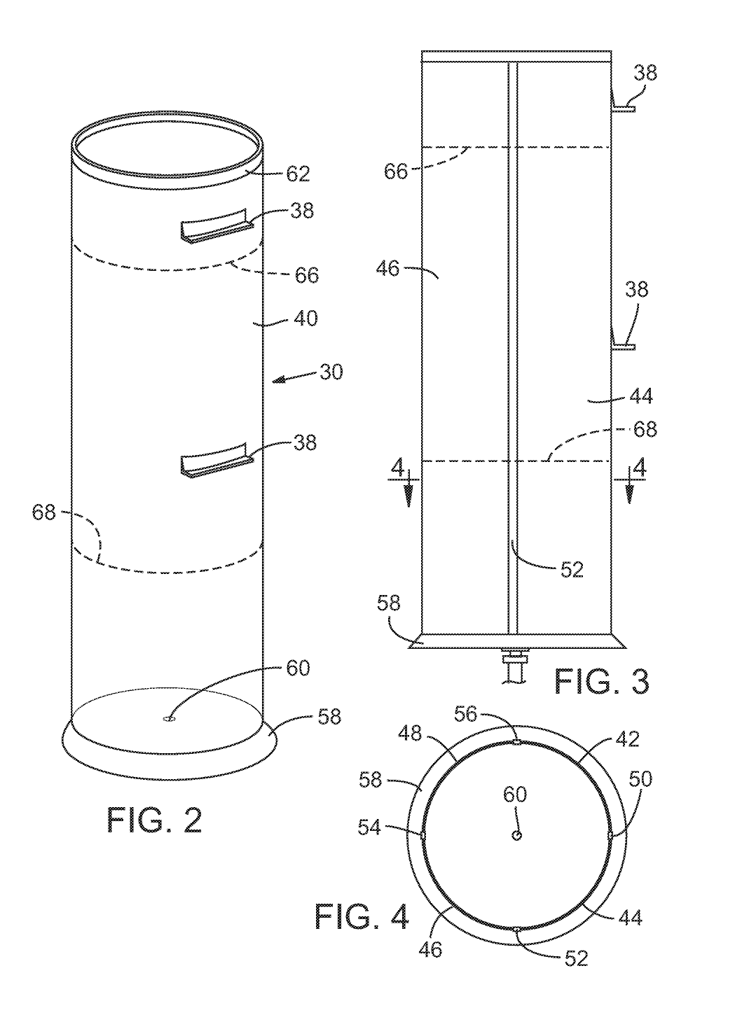

Each tank 30 is fabricated so that at least a substantial percentage of the cylindrical walls that define the tank are transparent or translucent so that sunlight passes through the walls of the tank and into the interior of the tank. There are many ways that a suitable tank 30 may be fabricated so that a substantial portion of the tank is transparent or translucent—the present invention is not limited to any particular manner of building the tank. Nonetheless, in a preferred embodiment and with reference to FIG. 2, the cylindrical wall 40 of tank 30 is fabricated of fiber reinforced composite material such as fiber reinforced plastic (FRP) that is about 0.090 inches in thickness. It has been found that this gauge FRP is sufficiently strong to provide structural integrity in a tank having a height to diameter ratio of 3:1, and a capacity of 5,000 gallons. There are many kinds of FRP and other composites available on the market that will suffice for use in building tanks 30. Most FRP materials comprise a polymer matrix that is reinforced with fibers, which commonly are fiberglass or aramid. Importantly, as noted, at least a substantial portion of the tank's walls must be transparent or translucent so that sunlight is transmitted through the walls. There are many kinds of translucent and transparent FRPs on the market and they are available from numerous sources; in terms of the amount of external light that is transmitted through the tank wall into the interior of the tank it is only necessary that sufficient light is transmitted that metabolic processes of the organisms contained in the tank are maintained. The relative degree of translucence or transparency is on that basis not essential so long as sufficient light is transmitted through the tank wall that the organisms can metabolize.

In the embodiment illustrated in FIGS. 3 and 4, tank 30 is fabricated from four separate elongate sections of FRP, labeled 42, 44, 46 and 48 in FIG. 4, and each of which defines an arc about the longitudinal axis of the section of about 90°. Each of these curved elongate sections is attached to adjacent sections at a seam, labeled 50, 52, 54 and 56 in FIG. 4. Each seam is watertight and preferably is an overlapping seam where the joined sections of FRP are welded or otherwise attached together (for example, by sonic welding or with appropriate adhesives). The cylinder is joined to a base 58 in a watertight fashion and the base includes an inlet/outlet port 60, which in FIG. 3 may be seen as connected to fittings 60 that lead to the transfer hose 24. The upper rim of tank 30 preferably but optionally includes a reinforcing rim 62 to stabilize and strengthen the tank. Reinforcing bands may also optionally be placed around tank 30 at desired locations along the length of the tank if desired in order to reinforce the tank. The brackets 38 are attached to the tank by welding or with adhesive.

The method of using tank 30 and the associated components to separate A. flos aquae from M. aeruginosa will now be described. Barge 16 is used to collect water populated with the target organism, A. flos aquae. As noted above, A. flos aquae is commercially harvested in Upper Klamath Lake in Oregon. In most instances, M. aeruginosa and a variety of other material is harvested with the raw material harvested by the barge. The raw material is an aqueous solution that contains the desire organism, and other organisms that must be separated from the A. flos aquae in order to further process the desired organism for human consumption. The raw material may be harvested in any number of ways, but typically suction pumps are used to collect the water and organisms into the tanks 18 and 20 on the barge 16. As might be expected, the raw material in the environment of the lake tends to spread out into “slicks.” The harvesting process involves picking up the entire slick, which usually contains A. flos aquae, M. aeruginosa, and a variety of foreign debris such as pine pollen and the like. The loaded barge 16 is docked at dock 12 and transfer hose 24 is fluidly connected to fittings 60. At this point, the water in tanks 18 and 20, which contains the target species A. flos aquae and other organisms, including M. aeruginosa, is pumped into the tanks 30a and 30b by operation of pump 22. The raw material enters the tanks through inlet/outlet 60. All constituents in the water are well and thoroughly mixed when the tank is filling due to the agitation caused by pumping and filling. Nonetheless, active mixing may optionally be used if desired.

Once the tanks 30a and 30b are filled they are allowed to rest without agitation. As noted earlier, both A. flos aquae and M. aeruginosa have gas vacuoles. These vacuoles begin to fill with gas as metabolic processes continue in the presence of sunlight and certain nutrients already present in the water drawn from the lake. Thus, in the presence of sunlight, which is transmitted through the walls of tanks 30 and into the water-containing interior, and the nutrients present in the water, organisms contained in the water continue metabolism according to the conditions in the tanks, resulting in gas production. Because the top end of the tanks are open, gas exchange with the atmosphere continues, as does a certain amount of evaporation. As the organism's vacuoles fill with gas, the organisms exhibit buoyancy properties according to the specific organism. Both A. flos aquae and M. aeruginosa rise vertically upwardly in the tank as the gas vacuoles fill. Because the axis of the tank is vertically oriented, the tank defines a spatially restricted environment and the normal tendency of the organisms to spread out into a relatively dispersed slick is restricted. The approximately spherically shaped M. aeruginosa colonies are much smaller than the thread-like colonies of A. flos aquae. In addition, the thread-like A. flos aquae colonies in the tank tend to orient with the thread axis perpendicular to the vertical direction. Both the larger size and dramatically different geometry of the A. flos aquae vacuoles give it a smaller vertical velocity (driven by the buoyant force on a colony) than the smaller, nearly spherical M. aeruginosa colonies. Also, under at least some light and nutrient conditions, some cyanobacteria and possibly A. flos aquae and/or M. aeruginosa have the ability to “self propel”, as described above. It is not known whether such propulsive forces are operating under the environmental conditions in the tank. It is, however, known that the net result of all the forces acting on the A. flos aquae and M. aeruginosa is that after a period of 5 to 24 hours, the M. aeruginosa colonies are mostly physically separated from the A. flos aquae and near the top of the tank while the A. flos aquae colonies are lower in the tank—in other words, the different organisms stratify in the tank and there is a distinct line of separation between the two organisms. This physical line of separation of the different organisms is shown in the figures with a schematic dashed line 66. The M. aeruginosa colonies are found vertically above line 66 and the A. flos aquae are found below line 66. Again due to the spatially restricted environment of the interior of the tank, the organisms are not only separated from one another but are also concentrated into well-defined masses where the colonies are in far greater concentration than would be found in a lake environment.

The tank is allowed to rest for a period of between 5 to 24 hours during which time the organisms stratify based on their buoyancy responses. The time required depends on factors such as the amount of available light, the temperature, and the nutrient density. At the point in time that the organisms are stratified and concentrated, the tank thus contains an upper layer of organisms that comprises primarily M. aeruginosa, although this layer also contains much of the foreign matter including pine pollen. The tank also contains an intermediate layer comprising the target organism A flos aquae that are concentrated, and a lower layer that is relatively clear water that is relatively free of any algae. The physical line of separation between the intermediate layer of A flos aquae and the lower layer of clear water is shown on the figures with the dashed line with reference number 68.

With the colonies separated in this manner, the more concentrated M. aeruginosa material in the top portion of the tank can be pumped out and the bottom portion containing little or no M. aeruginosa (i.e., the liquid below line 66) can be drained out via inlet/outlet 60. Preferably, the water in the tank above line 66 is aspirated out through the open top of the tank with suction pumps operated by workers on gangway 36, but in either case, the aqueous solution remaining in the tank once the material above line 66 and material below line 68 has been removed contains a concentrated level of the desired target organism A. flos aquae and substantially less M. aeruginosa than the original raw water placed in the tanks 30—the concentration of the target organism in the aqueous solution at this point is between 5 and 50 percent higher than it was in the original aqueous solution. This process may be repeated until the M. aeruginosa content of the harvested material is acceptably low that the water containing A. flos aquae may be further processed, ultimately for human consumption.

As noted previously, in the illustrated embodiment substantially the entire wall of tank 30 is transparent or translucent. The invention is not limited to any particular percentage of the tank being transparent or translucent, or having light-transmitting characteristics. As a general rule, the greater the percentage of the tank wall that transmits light, the higher the efficiency of the separation. It will be appreciated that there are many ways to make a tank with either fully or partially transparent walls. Moreover, the principal of the invention as characterized in the claims contemplates separation of A. flos aquae from M. aeruginosa by reliance upon the different rates of buoyancy of the organisms under similar conditions. The invention thus contemplates use of artificial light as well as sunlight.

It will further be appreciated that the design of tank 30 may be altered substantially without departing from the scope of the invention. For example, the locations of drains, ports, etc. may be varied according to the needs of a particular location.

Besides relying upon the different buoyancy responses between A. flos aquae from M. aeruginosa to separate the organisms in raw water, other factors may be manipulated in the separation tanks 30 in order to make the rate of separation more efficient. For example, the amount and intensity of light to which the tanks are exposed, the wavelength of the light, the water temperature, and the presence and concentration of nutrients such as phosphorous and nitrogen in the water may be altered in order to further affect the relative buoyancy responses of the two organisms. For example, the elasticity of the gas vesicle membrane of the organisms is influenced by the temperature and the available nutrients in the water. As such, these factors play a role in determination of how the cyanobacteria respond to an applied pressure, which could be the hydrostatic pressure at different depths in the tanks. The invention thus contemplates regulation of the pressure in the tanks in addition to temperature and nutrient conditions of the water as a factor influencing isolation of the desired A. flos aquae organisms.

Finally, physically altering or disrupting gas vacuoles has an affect on the buoyancy. Ultrasound cavitation is known to collapse the gas vacuoles of cyanobacteria. Since the gas vacuole structure varies considerably between A. flos aquae or M. aeruginosa, using ultrasonic cavitation at controlled frequency and power combinations may be used to selectively change the buoyancy of either A. flos aquae or M. aeruginosa and thereby allow the separation of one from the other.

While the present invention has been described in terms of a preferred embodiment, it will be appreciated by one of ordinary skill that the spirit and scope of the invention is not limited to those embodiments, but extend to the various modifications and equivalents as defined in the appended claims.

I claim:

1. A method for separating A. flos aquae from M. aeruginosa in an aqueous solution containing both organisms, comprising the steps of:
a) providing a tank for containing the aqueous solution, said tank having a vertically oriented axis and an open top, and providing said tank with at least a portion of its tank wall configured for transmitting light from the exterior of the tank to the tank interior;
b) filling the tank with the aqueous solution containing A. flos aquae and M. aeruginosa;
c) exposing the tank to light so that both A. flos aquae and M. aeruginosa in the tank produce gas and said gas fills gas vacuoles in said organisms to generate a buoyancy response;
d) allowing the A. flos aquae to separate from A. flos aquae from M. aeruginosa in the tank according to the buoyancy response.

2. The method according to claim 1 in which the A. flos aquae stratifies from the M. aeruginosa at a different vertical level in the tank.

3. The method according to claim 2 in which the A. flos aquae is vertically below the M. aeruginosa in the tank.

4. The method according to claim 3 including the steps of allowing the aqueous solution to rest while exposed to light and allowing the organisms to metabolize to thereby generate gas.

5. The method according to claim 4 wherein the buoyancy response includes said cyanobacteria rising vertically in the tank, and including the step of allowing the A. flos aquae to rise at a slower rate than the M. aeruginosa.

6. The method according to claim 5 including the step of removing a selected one of either the A. flos aquae or M. aeruginosa from the tank.

7. The method according to claim 6 including further processing the A. flos aquae into food for human consumption.

8. The method according to claim 1 including varying the temperature of the water in the tank to modify the buoyancy response.

9. The method according to claim 1 including adding nutrients to the water to modify the buoyancy response.

10. A method for separating A. flos aquae from M. aeruginosa, comprising the steps of:
a) placing an aqueous solution in a container having an open top and a vertically oriented axis, the aqueous solution containing at least A. flos aquae and M. aeruginosa;
b) exposing the aqueous solution to light so that the A. flos aquae and M. aeruginosa experience the same environmental conditions;
c) allowing the A. flos aquae and M. aeruginosa to metabolize and to thereby produce gas; and
d) allowing the A. flos aquae and M. aeruginosa to stratify in the aqueous solution based on differing buoyancy between the two species of cyanobacteria.

11. The method according to claim 10 including the step of causing one of the either A. flos aquae or M. aeruginosa to rise in the aqueous solution at a faster rate than the other of the two species.

12. The method according to claim 11 wherein the species that rises at a faster rate is M. aeruginosa.

13. The method according to claim 12 wherein A. flos aquae is located in a layer stratified in the container below the M. aeruginosa.

14. The method according to claim 10 including the step of removing the M. aeruginosa from the container, and repeating steps b through d of claim 10 until concentration of mycrocystin in the container is less than 1 PPM.

15. Apparatus for separating A. flos aquae from M. aeruginosa, comprising:
a cylindrical tank having a vertical axis, a tank wall, an open top, an inlet into the tank, and an outlet from the tank, wherein at least a substantial portion of the tank wall is transparent, and wherein A. flos aquae and M. aeruginosa contained in the tank in water produce gas and said gas causes the different species to rise to different levels in the tank.

16. The apparatus according to claim 16 wherein the entire tank wall is transparent.

17. The apparatus according to claim 17 wherein the tank has a height and a diameter, and the ratio of the height to diameter is about 3:1.

18. The apparatus according to claim 18 wherein the tank comprises fiber reinforced plastic.

19. The apparatus according to claim 15 including a pump fluidly connected to the inlet for introducing water and the A. flos aquae and M. aeruginosa into the tank.

20. The apparatus according to claim 15 used to separate A. flos aquae from M. aeruginosa.
